(19) United States
(12) Patent Application Publication
(10) Pub. No.: US 2026/0137343 A1
(43) Pub. Date: May 21, 2026

(54) INTERACTION APPARATUS OF WRIST-WORN DEVICE AND WRIST-WORN DEVICE

(71) Applicant: GOERTEK TECHNOLOGY CO., LTD, Qingdao, Shandong (CN)

(72) Inventors: Jianlei REN, Qingdao, Shandong (CN); Junjie PAN, Qingdao, Shandong (CN); Huan LI, Qingdao, Shandong (CN)

(21) Appl. No.: 19/114,482

(22) PCT Filed: Oct. 24, 2023
(86) PCT No.: PCT/CN2023/126152

§ 371 Date: Mar. 24, 2025

(30) Foreign Application Priority Data
Oct. 31, 2022 (CN) 202211352759.7
Jul. 11, 2023 (CN) 202310847344.5

(51) Int. Cl. A61B 5/00; A61B 7/00; A61B 7/04

(52) U.S. Cl. CPC A61B 5/681; A61B 7/003; A61B 7/04; A61B 2562/0204

(57) ABSTRACT

Disclosed are an interaction apparatus of wrist-worn device and a wrist-worn device. The interaction apparatus of wrist-worn device includes a main structure including a housing and a mainboard disposed on the housing, and a signal acquisition portion disposed protruding from a side peripheral surface of the housing, the signal acquisition portion includes a physiological sound sensor used for detecting heart sound signals or lung sound signals. The physiological sound sensor collects a vibration signal of the heart or lung and transmits the signal to the mainboard, the mainboard processes the signal to monitor the heart or lung. According to the present disclosure, the cost of replacing the wristband is reduced; the original inner structure of the housing is prevented from being modified; and when the hand is lifted, the signal acquisition portion can be directly attached to the heart position or the lung position for signal detection.

The present application claims priority to the Chinese Patent Application No. 202211352759.7, entitled “Interaction Apparatus of Wrist-worn Device and Wrist-worn Device”, filed with the China National Intellectual Property Administration on Oct. 31, 2022, and the Chinese Patent Application No. 202310847344.5, entitled “Interaction Apparatus of Wrist-worn Device and Wrist-worn Device”, submitted to the China Patent Office on Jul. 11, 2023, the entire contents of which are incorporated herein by reference.

### TECHNICAL FIELD

The present disclosure relates to the technical field of wearable device, in particular, relates to an interaction apparatus of wrist-worn device and a wrist-worn device.

### BACKGROUND ART

The development of science and technology is more and more rapid, especially intelligent wearable products, in recent years, are more and more abundant in functions, more and more humanized and more attractive in appearance.

Health detection has always been an important direction for research of intelligent wearable products, for example, health monitoring of existing intelligent wearable products can detect heart rate without detection of heart sounds or lung sounds, a traditional device for detecting heart sounds or lung sounds is generally a stethoscope, the stethoscope is generally a medical device commonly used by doctors, and it is difficult for ordinary people to carry the stethoscope so as to measure heart and lung positions at any time, therefore it is necessary to provide a device convenient to operate so as to measure heart sounds or lung sounds at any time.

### SUMMARY

A main purpose of the present disclosure is to provide an interaction apparatus of wrist-worn device, which is convenient to carry and operate and is used for detecting heart sounds or lung sounds.

In order to achieve the above purpose, the present disclosure provides an interaction apparatus of wrist-worn device, including:
a main structure including a housing and a mainboard disposed on the housing; and
a signal acquisition portion protruding from a side peripheral surface of the housing, the signal acquisition portion including a physiological sound sensor configured to detect heart sound signals or lung sound signals.

Optionally, a mounting portion is formed by protruding outwards from the side peripheral surface of the housing, the mounting portion is provided with an avoidance groove, the signal acquisition portion is disposed on the mounting portion, and the physiological sound sensor is disposed facing the avoidance groove.

Optionally, an avoidance hole is formed at a bottom of the avoidance groove, and the avoidance hole communicates with an inner cavity of the housing.

Optionally, the housing is defined to have two end surfaces and the side peripheral surface connecting the two end surfaces, and an angle between a surface of the mounting portion and a surface of the housing is an obtuse angle.

Optionally, the signal acquisition portion includes a cover plate, the physiological sound sensor is disposed at an inner side of the cover plate, and the cover plate is disposed to cover a surface of the mounting portion.

Optionally, the inner side of the cover plate is provided with a mounting groove, the physiological sound sensor is mounted in the mounting groove, an avoidance hole is formed at a bottom of the avoidance groove, and the physiological sound sensor is disposed in the avoidance hole on a side away from the cover plate.

Optionally, a wristband connecting portion is formed by protruding outwards from the side peripheral surface of the housing, the wristband connecting portion is disposed on a side edge of the mounting portion, and the cover plate is connected to the wristband connecting portion.

Optionally, a waterproof structure is provided between the cover plate and the wristband connecting portion;
a signal transmission structure is further provided on the housing, one end of the signal transmission structure is electrically connected to the signal acquisition portion, and the other end of the signal transmission structure is electrically connected to the mainboard.

Optionally, an adhesive film is provided on a surface of the wristband connecting portion, and the cover plate is adhered to the wristband connecting portion; and/or
a gap is formed between the cover plate and a surface of the mounting portion; and/or
the interaction apparatus of wrist-worn device further includes a wristband, the wristband is disposed on the wristband connecting portion, and the signal acquisition portion is disposed between the housing and the wristband; and/or
the other end of the signal transmission structure is provided with one of a buckle and a slot, and the mainboard is provided with the other of the buckle and the slot; and/or
the signal transmission structure is a flexible circuit board; and/or
the housing is symmetrically provided with two signal acquisition portions, and at least one of the signal acquisition portions is provided with the physiological sound sensor therein.

Optionally, the physiological sound sensor includes at least one of a bone conduction sensor, a microphone, an acceleration sensor, an inertial sensor, a piezoelectric ceramic, a pressure sensor, and a vibration detection sensor; and/or
the number of the physiological sound sensor is at least one.

The present disclosure provides a wrist-worn device, including the interaction apparatus of wrist-worn device and a wristband, wherein the wristband is connected to the interaction apparatus of wrist-worn device; and
the wrist-worn device is a watch or a bracelet.

Optionally, the wrist-worn device is a watch, the main structure includes a dial and a housing, the dial is disposed in the housing, and the signal acquisition portion is disposed close to a 6 o'clock position and/or a 12 o'clock position of the dial.

The present application provides an interaction apparatus of wrist-worn device, the interaction apparatus of wrist-worn device includes a main structure and a signal acquisition portion, the main structure includes a housing and a mainboard disposed on the housing, and the signal acquisition portion is disposed protruding from a side peripheral surface of the housing; the signal acquisition portion includes a physiological sound sensor, the physiological sound sensor is used for detecting heart sound signals or lung sound signals. Vibration signals of the heart or the lung are collected through the signal acquisition portion and transmitted to the mainboard, and the mainboard processes the vibration signals so as to monitor the condition of the heart or the lung. The signal acquisition portion is disposed on the housing so as to avoid the signal acquisition portion from being disposed on the wristband, thereby reducing the cost of replacing the wristband; in the meanwhile, the signal acquisition portion is disposed protruding from the side peripheral surface of the housing so as to avoid an original internal structure of the interaction apparatus of wrist-worn device from being modified, thereby reducing the design cost; and the protrusion of the signal acquisition portion from the side peripheral surface of the housing facilitates, when a hand is lifted, direct attachment of the signal acquisition portion to the heart position or the lung position for signal monitoring.

### BRIEF DESCRIPTION OF DRAWINGS

In order to more clearly illustrate the embodiments of the present disclosure or the technical solutions in the prior art, the accompanying drawings that need to be used in the description of the embodiments or the prior art will be briefly described below, and obviously, the accompanying drawings in the following description are only some embodiments of the present disclosure, and for those skilled in the art, other accompanying drawings may be obtained according to the structures shown in these accompanying drawings without creative efforts.

FIG. 1 is a schematic structural diagram of an embodiment of an interaction apparatus of wrist-worn device according to the present disclosure;

FIG. 2 is an exploded schematic structural diagram of the interaction apparatus of wrist-worn device in FIG. 1;

FIG. 3 is an exploded schematic structural diagram of the interaction apparatus of wrist-worn device in FIG. 1;

FIG. 4 is a schematic structural diagram of a section of the interaction apparatus of wrist-worn device in FIG. 1;

FIG. 5 is a schematic structural diagram of the housing;

FIG. 6 is a schematic structural diagram of the cover plate;

FIG. 7 is a schematic structural diagram of an embodiment of an acquisition module according to the present disclosure.

### DESCRIPTION OF REFERENCE NUMBER

| Reference | | Reference | |
|---|---|---|---|
| Number | Name | Number | Name |
| 100 | interaction apparatus | 35 | wristband connecting |
| | of wrist-worn device | | portion |
| 10 | main structure | 50 | wristband |
| 11 | mainboard | 6 | bone conduction sensor |
| 13 | housing | 61 | magnet |
| 131 | mounting portion | 62 | coil |
| 1310 | avoidance groove | 63 | first vibration panel |
| 1310a | avoidance hole | 64 | second vibration panel |
| 20 | adhesive film | 65 | elastic connector |
| 30 | signal acquisition | 66 | adhesive layer |
| | portion | | |
| 31 | cover plate | 67 | vibration transmission |
| | | | layer |
| 311 | mounting groove | 70 | signal transmission |
| | | | structure |
| 33 | physiological sound | 90 | connecting terminal |
| | sensor | | |

### DETAILED DESCRIPTIONS

The following clearly and completely describes the technical solutions in the embodiments of the present disclosure with reference to the accompanying drawings in the embodiments of the present disclosure, and obviously, the described embodiments are only a part of the embodiments of the present disclosure, but not all the embodiments. Based on the embodiments of the present disclosure, all other embodiments obtained by those skilled in the art without creative efforts fall within the protection scope of the present disclosure.

It should be noted that all directional indications (such as up, down, left, right, front, and rear) in the embodiments of the present disclosure are merely used to explain a relative position relationship and a movement condition and the like between components in a specific posture (as shown in the accompanying drawings), and if the specific posture changes, the directional indication changes accordingly.

In the present disclosure, unless otherwise explicitly specified and defined, terms “connected”, “fixed” and the like should be understood broadly, for example, “fixed” may be a fixed connection, a detachable connection, or an integrated connection; may be a mechanical connection, or an electrical connection; may be a direct connection, or an indirect connection through an intermediate medium, may be an internal communication between two elements or an interaction relationship between two elements, unless otherwise explicitly defined. For those skilled in the art, the specific meaning of the above terms in the present disclosure can be understood according to specific conditions.

In addition, in the present disclosure, descriptions such as “first”, “second” and the like are merely used for description purposes, and cannot be understood as indicating or implying relative importance thereof or implicitly indicating a quantity of indicated technical features. Thus, a feature defining with “first” and “second” may explicitly or implicitly include at least one of the features. In addition, “and/or” appearing throughout the text means that three parallel solutions are included, for example, “A and/or B” includes A solution, B solution, or solution that satisfies A and B simultaneously. In addition, the technical solutions among the various embodiments may be combined with each other, but must be based on the fact that the technical solutions can be realized by those of ordinary skill in the art, and when the combination of the technical solutions contradicts or cannot be realized, it should be considered that the combination of the technical solutions does not exist or fall within the protection scope claimed by the present disclosure.

The development of science and technology is more and more rapid, especially intelligent wearable products, in recent years, are more and more abundant in functions, more and more humanized and more attractive in appearance.

Health detection is always an important direction of research on intelligent wearable products, and if the intelligent wearable products can be used to collect or monitor heart sounds or lung sounds, great convenience is provided to users who have requirements, and the intelligent wearable products can collect or detect anytime and anywhere, so that the user can know their health conditions in time.

In order to meet the requirement that the user wants to know the health condition of the heart or the lung at any time, the present application provides an interaction apparatus of wrist-worn device, and aims to provide an interaction apparatus of wrist-worn device convenient to carry and operate for detecting the vibration of the heart or the lung.

The interaction apparatus of wrist-worn device 100 includes a main structure 10 and a signal acquisition portion 30, wherein the main structure 10 includes a housing 13 and a mainboard 11 disposed on the housing 13, and the signal acquisition portion 30 is disposed protruding from a side peripheral surface of the housing 13; the signal acquisition portion 30 includes a physiological sound sensor 33, and the physiological sound sensor 33 is used for detecting a heart sound signal or a lung sound signal, wherein the heart sound signal or the lung sound signal refers to vibration data caused by heartbeat or respiration of the user, and the vibration data comes from the heart sounds or the lung sounds.

Physiological sound refers to sound generated by mechanical wave phenomenon in human organs such as heart and lung.

The physiological sound sensor 33 may be a combination of one or more of a bone conduction sensor, a microphone, an acceleration sensor, an inertial sensor, a piezoelectric ceramic, a pressure sensor, or a vibration detection sensor, wherein the number of each type of sensors may be one or more, or multiple sensors may be used in combination, for example, the physiological sound sensor 33 may be two bone conduction sensors, or two piezoelectric ceramics, or a combination of one bone conduction sensor and one acceleration sensor.

In general, the main structure 10 and a wristband 50 are two detachable parts, and compared with the main structure, the wristband is cheap in price, and in the case that the wristband is damaged, the wristband is generally selected to be replaced, therefore, if an important functional device is provided on the wristband, then at the time of replacing the wristband, the cost of replacing the wristband is increased due to the added price of the functional device, for example, if the physiological sound sensor 33 is provided on the wristband, on one hand, due to the structure that the physiological sound sensor 33 is electrically connected with the mainboard in the main structure, it is inconvenient to detach the physiological sound sensor, and on the other hand, the replacement cost is increased, therefore, by means of directly providing the physiological sound sensor 33 on the main structure, the user can conveniently replace the wristband.

It can be understood that the wrist-worn device refers to a device that can be worn on an arm, for example, the wrist-worn device may be worn on an arm, or may be worn on a wrist, or may be worn on a neck, an ankle, or a leg for convenience, which is not specifically limited.

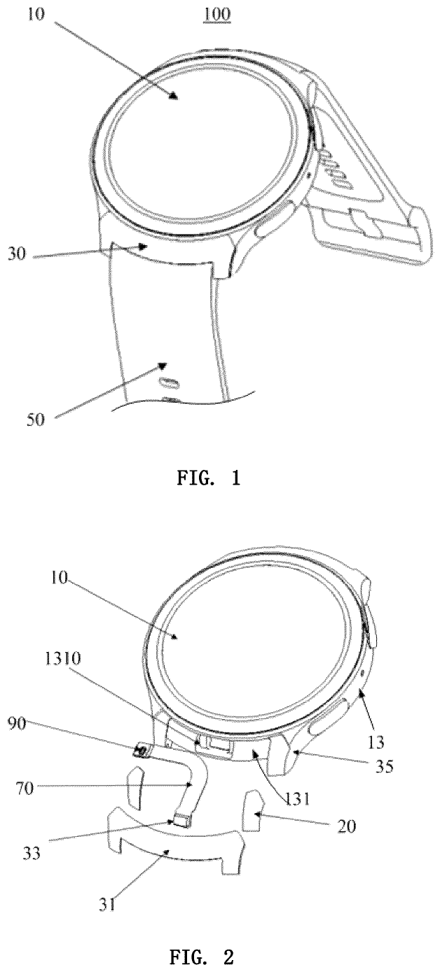

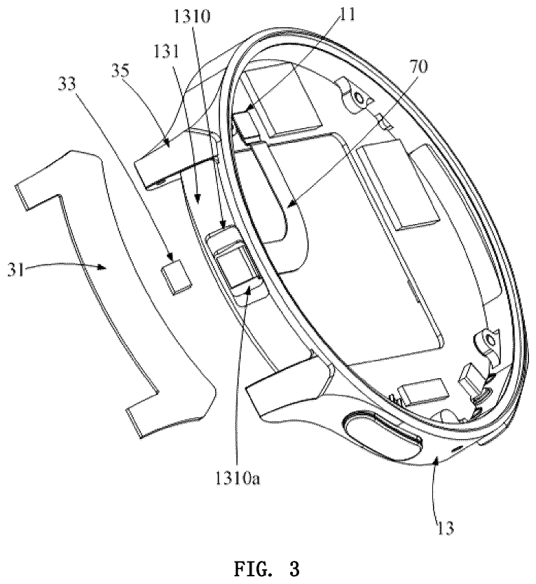

As shown in FIG. 1 and FIG. 2, a vibration signal of the heart or lung is collected and transmitted to the mainboard 11 by the signal acquisition portion 30, and the mainboard 11 processes the vibration signal to monitor the condition of the heart or lung. The signal acquisition portion 30 is disposed on the housing 13, so as to avoid the signal acquisition portion 30 from being disposed on the wristband, thereby reducing the cost of replacing the wristband; in the meanwhile, the signal acquisition portion 30 is disposed protruding from the side peripheral surface of the housing 13, so as to avoid the original internal structure of the interaction apparatus of wrist-worn device from being modified, thereby reducing the design cost; and the protrusion of the signal acquisition portion 30 from the side peripheral surface of the housing 13 facilitates, when a hand is lifted, direct attachment of the signal acquisition portion 30 to the heart position or the lung position for signal monitoring. Moreover, the structure of the interaction apparatus of wrist-worn device is designed to form a new structural style.

The physiological sound sensor in the present disclosure is used to receive the vibration signal of heart or lung of human body, and the bone conduction is one type of sound conduction manners, in which sound is converted into mechanical vibration of different frequencies, and sound waves are transmitted through skull, bony labyrinth, inner ear lymphatic fluid, spirochetes, auditory nerve and auditory center of human body.

Further, a mounting portion 131 is formed by protruding outwards from the side peripheral surface of the housing 13, the mounting portion 131 is provided with an avoidance groove 1310, the signal acquisition portion 30 is disposed on the mounting portion 131, and the physiological sound sensor 33 is disposed facing the avoidance groove 1310.

As shown in FIG. 2 to FIG. 4, the housing 13 serves to bear loading, an interior of the housing 13 is used for accommodating electrical devices, for example, the mainboard 11 is disposed in an inner cavity of the housing 13, and in order to avoid the signal acquisition portion 30 from being disposed in the inner cavity of the housing 13, so as to avoid new structural design and modification on the interior of the housing 13, the side peripheral surface of the housing 13 protrudes outward to form the mounting part 131, and the signal acquisition portion 30 is disposed on the mounting part 131, moreover, the mounting part 131 is provided with the avoidance groove 1310, the signal acquisition portion 30 is disposed on the mounting part 131, and the physiological sound sensor 33 is disposed facing the avoidance groove 1310. In this way, the physiological sound sensor 33 does not contact the mounting part 131, and the physiological sound sensor 33 is prevented from being interfered during the signal acquisition process.

It can be understood that, considering the requirement of the structure of the physiological sound sensor 33 itself, the physiological sound sensor 33 has a vibration structure, and the avoidance groove 1310 forms avoidance, so as to prevent the physiological sound sensor 33 from contacting the mounting portion 131 and correspondingly affecting the reception and transmission of the vibration structure signal.

For example, the physiological sound sensor 33 is a bone conduction loudspeaker 6, the bone conduction loudspeaker 6, as shown in FIG. 7, includes a magnet 61, a coil 62, a first vibration panel 63, a second vibration panel 64, an elastic connector 65, an adhesive layer 66, and a vibration transmission layer 67; the magnet 61 is disposed on an inner side of the first vibration panel 63, the coil 62 is disposed on an inner side of the second vibration panel 64, one end of the elastic connector 65 is connected to the first vibration panel 63, the other end of the elastic connector 65 is connected to the second vibration panel 64, and a loudspeaker cavity is formed by connecting the first vibration panel 63, the elastic connector 65, and the second vibration panel 64; the adhesive layer 66 is disposed on an outer side of the second vibration panel 64, and the vibration transmission layer 67 is connected with the adhesive layer 66; the vibration transmission layer 67 is an arc-shaped structure, and the arc-shaped structure can realize excellent contact between the loudspeaker and the wrist, and comfort, and can optimize the effect of loudspeaker bone conduction.

Further, an avoidance hole 1310a is formed at the bottom of the avoidance groove 1310, and the avoidance hole 1310a communicates with the inner cavity of the housing 13.

As shown in FIG. 3, in order to further prevent the physiological sound sensor 33 from contacting a wall of the avoidance groove 1310, an avoidance hole 1310a is formed at the bottom of the avoidance groove 1310, and the avoidance hole 1310a communicates with the inner cavity of the housing 13, so that the physiological sound sensor 33 may not contact the bottom of the avoidance groove 1310 during the process of receiving the vibration signal, and in the meanwhile, it is also convenient for the physiological sound sensor 33 to be electrically connect to the mainboard 11 in the housing 13 through the avoidance hole 1310a.

Further, the housing 13 is defined to have two end surfaces and a side peripheral surface connecting the two end surfaces, and an angle formed by the surface of the mounting portion 131 and the surface of the housing 13 is an obtuse angle.

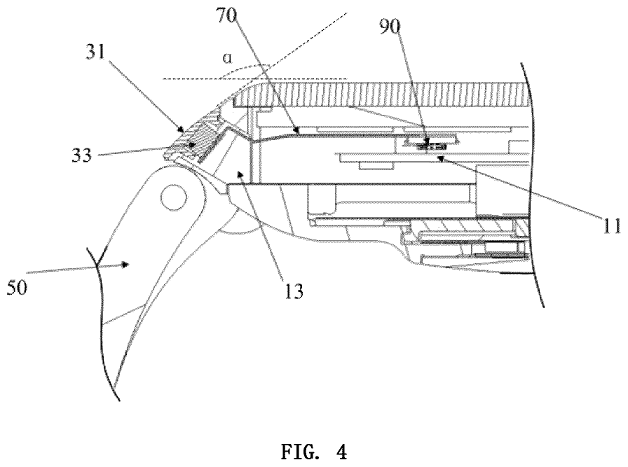

As shown in FIG. 4, in order for facilitating the attachment of the signal acquisition portion 30 to the heart or lung of the chest to monitor the heart or lung, the angle formed by the surface of the mounting portion 131 and the surface of the housing 13 is an obtuse angle, and as shown in FIG. 4, the angle α is an obtuse angle, so that the signal acquisition portion 30 protrudes towards the outer peripheral surface of the housing 13 and is obliquely disposed on the outer peripheral surface of the housing 13, thereby facilitating the attachment of the signal acquisition portion 30 to the heart or lung for detection.

Further, the signal acquisition portion 30 includes a cover plate 31, the physiological sound sensor 33 is disposed on an inner side of the cover plate 31, and the cover plate 31 is disposed to cover the surface of the mounting portion 131.

As shown in FIG. 2 to FIG. 4, in order to facilitate the physiological sound sensor 33 to receive the vibration signal of heart or lung, the signal acquisition portion 30 includes a cover plate 31, the physiological sound sensor 33 is disposed on an inner side of the cover plate 31, and the cover plate 31 is disposed to cover the surface of the mounting portion 131, that is, the cover plate 31 is disposed on the surface of the mounting portion 131. In a process of detecting heart sounds or lung sounds by using the interaction apparatus of wrist-worn device 100, the interaction apparatus of wrist-worn device 100 is placed on the heart or the lung of the chest, the cover plate 31 is attached to heart or lung, the cover plate 31 receives the vibration signal of heart or lung earliest, and the received signal is transmitted to the physiological sound sensor 33 through the cover plate 31; since the cover plate 31 is in contact with the physiological sound sensor 33, the vibration signal is transmitted to the physiological sound sensor 33 through the cover plate 31, that is, audio acquisition is completed. Moreover, in order to effectively receive the vibration signal, as shown in FIG. 3, the cover plate 31 completely covers the mounting portion 131, so that the cover plate 31 has a large area, and the cover plate 31 with a large area can cover the heart region or the lung region of human body, thereby ensuring accuracy and integrity of receiving the vibration signal.

Further, the inner side of the cover plate 31 is provided with a mounting groove 311, the physiological sound sensor 33 is mounted in the mounting groove 311, an avoidance hole 1310a is formed at the bottom of the avoidance groove 1310, and the physiological sound sensor 33 is disposed in the avoidance hole 1310a on a side away from the cover plate 31.

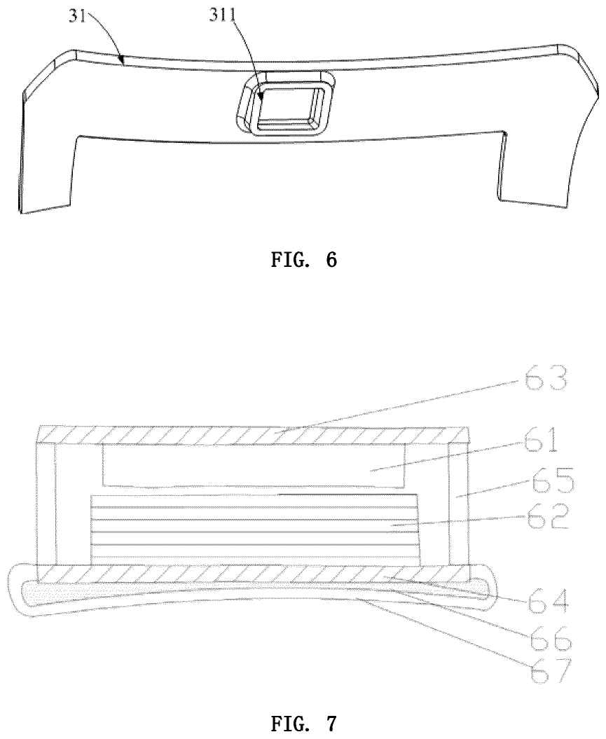

As shown in FIG. 4 and FIG. 6, in order to ensure the stability of mounting the physiological sound sensor 33, a mounting groove 311 is provided on the inner side of the cover plate 31, and the physiological sound sensor 33 is mounted in the mounting groove 311, so that the physiological sound sensor 33 may not fall off due to vibration in the process of transmitting the vibration signal to the physiological sound sensor 33 by the cover plate 31, and it can also be understood that the physiological sound sensor 33 may be connected in the mounting groove 311 in a snap-fit manner, or may be bonded in the mounting groove 311 by adhesive, which is not specifically limited. Preferably, the physiological sound sensor 33 is mounted in the mounting groove 311 by glue, so that the physiological sound sensor 33 is tightly connected to the cover plate 31, thereby reducing errors and losses in transmission of the vibration signal.

Moreover, in order to prevent the physiological sound sensor 33 from contacting the mounting portion 131, the physiological sound sensor 33 is disposed in the avoidance hole 1310a on a side away from the cover plate 31, so that the physiological sound sensor 33 may not contact the mounting portion 131 during vibration, thereby ensuring the quality of the vibration signal.

Further, a wristband connecting portion 35 is formed by protruding outwards from the side peripheral surface of the housing 13, the wristband connecting portion 35 is disposed on a side edge of the mounting portion 131, and the cover plate 31 is connected to the wristband connecting portion 35.

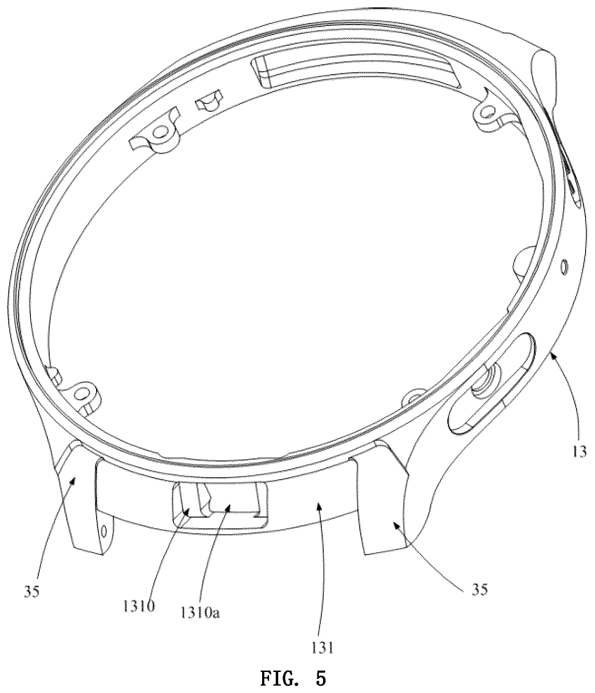

As shown in FIG. 2, FIG. 3 and FIG. 5, in order to facilitate the connection of the wristband 50, a wristband connecting portion 35 is formed by protruding outwards from the side peripheral surface of the housing 13, it can be understood that the mounting portion 131 is also a part of the housing 13, and the wristband connecting portion 35 may be directly formed by protruding outwards from the side peripheral surface of the housing 13, or may be formed by protruding outwards from the mounting portion 131, which is not specifically limited.

Moreover, the wristband connecting portion 35 is disposed on the side edge of the mounting portion 131, so that the wristband connecting portion 35 and the mounting portion 131 are connected together, therefore the overall structural strength and stability are high, and in the meanwhile, it is also convenient for the wristband connecting portion 35 to contact the heart region or the lung region in the test process. Moreover, the cover plate 31 is connected to the wristband connecting portion 35, so as to further enlarge the arrangement area of the cover plate 31, facilitate the expand the area of the cover plate 31, and thus be convenient to receive the vibration signal. Preferably, the cover plate 31 covers the surface of the wristband connecting portion 35, so that the region of the cover plate 31 on the wristband connecting portion 35 is exposed, which facilitates the contact with the heart region or the lung region. Of course, the cover plate 31 may be connected to the inner side or other region on the outer side of the wristband connecting portion 35, and when the cover plate 31 is connected to the inner side of the wristband connecting portion 35, the vibration can also be transmitted, because the wristband connecting portion 35 can receive the vibration information and directly transmit the vibration information to the cover plate 31 when the wristband connecting portion 35 contacts the heart region or the lung region.

Further, a waterproof structure is disposed between the cover plate 31 and the wristband connecting portion 35, and the housing 13 is further provided with a signal transmission structure 70, one end of the signal transmission structure 70 is electrically connected to the signal acquisition portion 30, and the other end of the signal transmission structure 70 is electrically connected to the mainboard 11.

In order to prevent water from entering the inner side of the cover plate 31 from between the cover plate 31 and the wristband connecting portion 35, a waterproof structure is provided between the cover plate 31 and the wristband connecting portion 35, and the waterproof structure may be a sealing gasket or a sealant, which is not specifically limited.

In order to electrically connect the physiological sound sensor 33 to the mainboard 11, the housing 13 is further provided with a signal transmission structure 70, one end of the signal transmission structure 70 is electrically connected to the signal acquisition portion 30, and the other end of the signal transmission structure 70 is electrically connected to the mainboard 11.

Further, the surface of the wristband connecting portion 35 is provided with an adhesive film 20, and the cover plate 31 is adhered to the wristband connecting portion 35; and/or, a gap is formed between the cover plate 31 and the surface of the mounting portion 131; and/or, the interaction apparatus of wrist-worn device 100 further includes a wristband 50, the wristband 50 is disposed on the wristband connecting portion 35, and the signal acquisition portion 30 is disposed between the housing 13 and the wristband 50; and/or, the other end of the signal transmission structure 70 is provided with one of a buckle and a slot, and the mainboard 11 is provided with the other of the buckle and the slot; and/or, the signal transmission structure 70 is a flexible circuit board; and/or, the physiological sound sensor 33 is a physiological sound sensor; and/or, the housing 13 is symmetrically provided with two signal acquisition portions 30, and at least one of the signal acquisition portions 30 is provided with a physiological sound sensor 33 therein.

The adhesive film 20 is provided on the surface of the wristband connecting portion 35, and the cover plate 31 is adhered on the surface of the wristband connecting portion 35, the arrangement of the adhesive film 20, on one hand, allows the cover plate 31 to be adhered on the surface of the wristband connecting portion 35, on the other hand, has a waterproof function.

Since the cover plate 31 is directly attached to the heart or the lung, the cover plate 31 also vibrates accordingly during the process of receiving the vibration signal of the heart or the lung, and in order to avoid weakening the vibration, the cover plate 31 is suspended on the mounting portion 131, so as to avoid weakening the vibration of the cover plate 31. It can be understood that, in order to form a gap between the cover plate 31 and the surface of the mounting portion 131, the surface of the mounting portion 131 and the surface of the wristband connecting portion 35 form a height difference, as shown in FIG. 5, and after the cover plate 31 is disposed to cover the surface of the mounting portion 131 and the surface of the wristband connecting portion 35, the cover plate 31 directly contacts the wristband connecting portion 35 and forms a gap with the surface of the mounting portion 131.

As shown in FIG. 1, the interaction apparatus of the wrist-worn device 100 further includes a wristband 50, the wristband 50 is disposed on the wristband connecting portion 35, the signal acquisition portion 30 is disposed between the main structure 10 and the wristband 50, the wristband 50 and the signal acquisition portion 30 are disposed along one direction of the main structure 10, so as to prevent a plurality of structures from protruding from the outer peripheral surface of the main structure 10, thereby reducing a size of the surface of the main structure 10; moreover, by means of disposing the signal acquisition portion 30 between the main structure 10 and the wristband 50, the signal acquisition portion 30 can be prevented from being collided.

It can be understood that, the signal transmission structure 70 is provided with one of a buckle and a slot on one end away from the physiological sound sensor 33, and the mainboard 11 is provided with the other of the buckle and the slot, and as shown in FIG. 2 and FIG. 4, the signal transmission structure 70 is provided with a connecting terminal 90, and the mainboard 11 is provided with a wiring port for plugging the connecting terminal, and the signal transmission structure 70 is electrically connected to the mainboard 11 in a plug-in manner, the common wiring terminal may be type-c, which is not specifically limited.

It can be understood that the signal transmission structure 70 is a flexible circuit board (FPC), so that the signal transmission structure 70 has a better flexibility when being bent.

As shown in FIG. 1, the housing 13 is symmetrically provided with two signal acquisition portions 30, and at least one of the signal acquisition portions 30 is provided with a physiological sound sensor 33 therein. That is, in appearance, the housing 13 is provided with two signal acquisition portions 30, and the housing 13 is more beautiful due to the symmetrical structure, it can be understood that one of the two signal acquisition portions 30 is provided with a physiological sound sensor 33, and of course, it is also possible that both the two signal acquisition portions 30 are provided with a physiological sound sensor 33, and the two physiological sound sensors 33 synchronously detect to improve the detection performance.

It can be understood that the wristband body 51 is made of a soft rubber material, so that wearing is more comfortable.

When collecting heart sounds or lung sounds, vibration generated by heart or lung of human body is directly transmitted to the cover plate 31 through clothes, and then transmitted to the physiological sound sensor 33 through the cover plate 31 so as to complete the data collection. During the collection process, since the physiological sound sensor 33 is fixed in the mounting groove 311, the mounting groove 311 is equivalent to a tympanic membrane which facilitates vibration transmission, and transmits the vibration of heart or lung to the physiological sound sensor 33.

As shown in FIG. 1, the cover plate 31 may be used as a collection region, and a motor or a loudspeaker is further disposed on the interaction apparatus of wrist-worn device 100, and the use method is as follows: when a user needs to collect the heart and lung sounds, the user lifts the wrist, and tightly attach the cover plate 31 on the housing 13 to the heart position or the lung position of human body, and after the collection is completed, the motor or the loudspeaker notifies the user that the collection is completed.

The assembly of the interaction apparatus of wrist-worn device 100 includes the following assembly process: attaching the physiological sound sensor 33 on one end of the FPC, the connecting terminal 90 being provided on the other end of the FPC, fixing the physiological sound sensor 33 in the mounting groove 311 of the cover plate 31, then fixing the cover plate 31 with the physiological sound sensor 33 on the housing 13 through the adhesive film 20, and finally connecting the end having the connecting terminal 90 to the mainboard 11 positioned in the housing 13, so that the physiological sound sensor 33 can be communicated with a microprocessor in the housing 13.

Further, the physiological sound sensor 33 includes at least one of a bone conduction sensor, a microphone, an acceleration sensor, an inertial sensor, a piezoelectric ceramic, a pressure sensor, and a vibration detection sensor; and/or, the number of the physiological sound sensor 33 is at least one.

The present application does not limit the type of the physiological sound sensor 33, as long as the physiological sound sensor 33 can meet the required functions, for example, including but not limited to collecting or monitoring heart sounds or lung sounds. The physiological sound sensor 3 includes but is not limited to at least one of a bone conduction sensor, a microphone, an acceleration sensor, an inertial sensor, a piezoelectric ceramic, a pressure sensor, and a vibration detection sensor.

The present disclosure does not limit the number of the physiological sound sensors 33, and at least one physiological sound sensor 33 is provided, which can be set according to actual needs. For example, the physiological sound sensor 33 includes one bone conduction sensor, or one microphone, or one acceleration sensor, or one inertial sensor, or one piezoelectric ceramic, or one pressure sensor, or one vibration detection sensor, or the physiological sound sensor 33 includes two physiological sound sensors, the two physiological sound sensors may be any combination of the bone conduction sensor, the microphone, the acceleration sensor, the inertial sensor, the piezoelectric ceramic, the pressure sensor, and the vibration detection sensor, for example, a combination of one bone conduction sensor and one microphone, and the like.

The present application also provides a wrist-worn device, the wrist-worn device includes the interaction apparatus of wrist-worn device 100 and the wristband 50, the wristband 50 is connected to the interaction apparatus of wrist-worn device, and the wrist-worn device is a watch or a bracelet. The interaction apparatus of wrist-worn device 100 achieves at least all beneficial effects brought by the technical solutions of the above embodiments since it adopts all the technical solutions of all the above embodiments, and details are not described herein again.

Further, the wrist-worn device is a watch, the main structure 10 includes a dial and a housing 13, the dial is disposed in the housing 13, and the signal acquisition portion 30 is disposed close to a 6 o'clock position and/or a 12 o'clock position of the dial.

When the human wrist faces the chest part of human body, generally, the palm center faces the chest part, thereby the most convenient action is to place the wrist to approach the chest part, and the positions of 6 o'clock and 12 o'clock in the dial are located on two sides of the wrist, therefore the positions of 6 o'clock and 12 o'clock in the dial can be more conveniently attached to the chest.

When collecting heart sounds or lung sounds, the signal acquisition portion 30 close to the 6 o'clock position is placed at the heart position or the lung position, vibration generated by the heart and the lung of human body is transmitted through the clothes to the cover plate 31 on which the physiological sound sensor 33 is mounted, and the vibration signal is transmitted to the physiological sound sensor 33 through the cover plate 31 since the cover plate 31 is connected with the physiological sound sensor 33, in this way, the audio collection is completed.

In the meanwhile, the signal acquisition portion 30 is disposed on the housing 13 to face the 6 o'clock position and the 12 o'clock position of the dial, the appearance after assembly has no difference from the structure at the 12 o'clock position on the other side, thus it has little influence on the appearance of the watch body, and it facilitates the user to operate the acquisition step by means of placing the signal acquisition portion 30 at this position.

The above are only preferred embodiments of the present disclosure, and are not intended to limit the patent scope of the present disclosure, and under the inventive concept of the present disclosure, any equivalent structural changes made by using the specification and the accompanying drawings of the present disclosure or direct/indirect use in other related technical fields are all included in the patent protection scope of the present disclosure.

1. An interaction apparatus of wrist-worn device, comprising:
a main structure comprising a housing and a mainboard disposed on the housing; and
a signal acquisition portion protruding from a side peripheral surface of the housing, wherein the signal acquisition portion comprises a physiological sound sensor configured to detect heart sound signals or lung sound signals.

2. The interaction apparatus of wrist-worn device according to claim 1, wherein a mounting portion is formed by protruding outwards from the side peripheral surface of the housing, the mounting portion is provided with an avoidance groove, the signal acquisition portion is disposed on the mounting portion, and the physiological sound sensor is disposed facing the avoidance groove.

3. The interaction apparatus of wrist-worn device according to claim 2, wherein an avoidance hole is formed at a bottom of the avoidance groove, and the avoidance hole communicates with an inner cavity of the housing.

4. The interaction apparatus of wrist-worn device according to claim 2, wherein the housing is defined to have two end surfaces and the side peripheral surface connecting the two end surfaces, and an angle between a surface of the mounting portion and a surface of the housing is an obtuse angle.

5. The interaction apparatus of wrist-worn device according to claim 2, wherein the signal acquisition portion comprises a cover plate, the physiological sound sensor is disposed at an inner side of the cover plate, and the cover plate is disposed to cover a surface of the mounting portion.

6. The interaction apparatus of wrist-worn device according to claim 5, wherein the inner side of the cover plate is provided with a mounting groove, the physiological sound sensor is mounted in the mounting groove, an avoidance hole is formed at a bottom of the avoidance groove, and the physiological sound sensor is disposed in the avoidance hole on a side away from the cover plate.

7. The interaction apparatus of wrist-worn device according to claim 5, wherein a wristband connecting portion is formed by protruding outwards from the side peripheral surface of the housing, the wristband connecting portion is disposed on a side edge of the mounting portion, and the cover plate is connected to the wristband connecting portion.

8. The interaction apparatus of wrist-worn device according to claim 7, wherein a waterproof structure is provided between the cover plate and the wristband connecting portion;
a signal transmission structure is further provided on the housing, one end of the signal transmission structure is electrically connected to the signal acquisition portion, and the other end of the signal transmission structure is electrically connected to the mainboard.

9. The interaction apparatus of the wrist-worn device according to claim 8, wherein an adhesive film is provided on a surface of the wristband connecting portion, and the cover plate is adhered to the wristband connecting portion; and/or
a gap is formed between the cover plate and a surface of the mounting portion; and/or
the interaction apparatus of wrist-worn device further comprises a wristband, the wristband is disposed on the wristband connecting portion, and the signal acquisition portion is disposed between the housing and the wristband; and/or
the other end of the signal transmission structure is provided with one of a buckle and a slot, and the mainboard is provided with the other of the buckle and the slot; and/or
the signal transmission structure is a flexible circuit board; and/or
the housing is symmetrically provided with two signal acquisition portions, and at least one of the signal acquisition portions is provided with the physiological sound sensor therein.

10. The interaction apparatus of wrist-worn device according to claim 1, wherein the physiological sound sensor comprises at least one of a bone conduction sensor, a microphone, an acceleration sensor, an inertial sensor, a piezoelectric ceramic, a pressure sensor, and a vibration detection sensor; and/or
the number of the physiological sound sensor is at least one.

11. A wrist-worn device, comprising the interaction apparatus of wrist-worn device according to claim 1 and a wristband, wherein the wristband is connected to the interaction apparatus of wrist-worn device, and
the wrist-worn device is a watch or a bracelet.

12. The wrist-worn device according to claim 11, wherein the wrist-worn device is a watch, the main structure comprises a dial and a housing, the dial is disposed in the housing, and the signal acquisition portion is disposed close to a 6 o'clock position and/or a 12 o'clock position of the dial.
